(19) United States
(12) Patent Application Publication
(10) Pub. No.: US 2007/0123891 A1
(43) Pub. Date: May 31, 2007

(54) Facet joint reamer

(75) Inventors: Wolfgang Ries, Linkenheim (DE); Mathias Notheis, Forst (DE)

Correspondence Address: SUGHRUE MION, PLLC, 2100 PENNSYLVANIA AVENUE, N.W., SUITE 800, WASHINGTON, DC, 20037, US

(73) Assignee: joimax GmbH

(21) Appl. No.: 11/585,867

(22) Filed: Oct. 25, 2006

(30) Foreign Application Priority Data
Oct. 26, 2005 (DE) 20 2005 016761.4

(51) Int. Cl. A61B 17/00

(52) U.S. Cl. 606/80

(57) ABSTRACT

In a facet joint reamer with a substantially cylindrical shank, teeth at the distal shank end and a handle at the proximal end, the shank and handle are separable.

### FIELD OF THE INVENTION

The invention relates to a facet joint reamer with a substantially cylindrical shank, with teeth provided at the distal shank end and with a handle at the proximal end.

### BACKGROUND OF THE INVENTION

Such a facet joint reamer is known from DE 699 17 683 T2. The known reamer has a hollow cylindrical shank, a handle at its rear, proximal end and teeth at its front end. Such a reamer is used for cutting out vertebral components in the vicinity of a lateral process of a vertebral column vertebra in order to create a postero-lateral access to the strangulated nerve roots of the central nervous system. Through said access removal then takes place of the intervertebral disk pulp tissue and other tissue types (capsular, cicatricial and ring tissue), because they press on the nerve roots. The vertebral process in question forms with an adjacent process of an adjacent vertebra the so-called facet joint.

Microinvasive surgery for the decompression of strangulated nerve roots using the known facet joint reamer is highly successful. However, it has been found that in the vicinity of the teeth, also in the case of standard cleaning and disinfection, osseous tissue can be left behind, which at best could be carefully removed with considerable effort. In addition, since reamers of different thicknesses are used, it would be desirable to be able to use different reamers with a common operating handle.

Therefore the problem of the invention is to improve such a facet joint reamer in the above-indicated manner.

### SUMMARY OF THE INVENTION

According to the invention this problem is solved by a facet joint reamer which is characterized in that the shank and handle are separable and in particular nondestructively. separable.

In a specific development the shank and handle are provided with a cooperating joining device, which is constructed in such a way that at its proximal end the shank has a lateral depression in which engages a ball seated on the handle and which in turn can be introduced into a recess on the reamer shank. The ball can be locked on the handle, so that as a result the reamer shank is held in securely seated manner in the handle. This results from the fact that a slider is movable between a release position for releasing the shank from the handle and a locking position in which the shank is reliably held in the handle.

According to a preferred development the slider is spring loaded, the slider being more particularly held in the locking position by a spring.

In a further development the slider has a widened, inner area into which the ball can retreat when the slider passes over the ball. This leads to a release position for the reamer shank and the latter can be removed from the handle in the indicated slider and ball positions.

Additionally, in a preferred development, the slider can be slid out of the locking position counter to the force of the spring by manual action into its release position and in accordance with the differing preferences of surgeons either the slider is slidable counter to the action of the spring in the proximal direction into its release position or the slider is slidable counter to the action of the spring in the distal direction into its release position.

Further developments of the invention are characterized in that the shank and handle are connectable to rotate with one another and in particular one connecting part of the shank is constructed as an external polygon, particularly trihedron and a reception area of the handle as an internal polygon, preferably an internal trihedron.

In a highly preferred development a connecting part of the shank is made from plastic, said connecting part being connected in a fixed, unreleasable (at least nondestructively release) manner with the reamer shank and is e.g. injection moulded around the same. This makes it possible to code different shank thicknesses, e.g. by differing colours of the plastic part, i.e. formation thereof with a reflection-absorption capacity in different wavelength ranges. In addition, on the plastic shank can be provided a marking, e.g. in the form of an elevation if the reamer shank is provided with a lateral protective lip projecting over the teeth for protection of nerves during working, said marking in the form of an elevation on the plastic part being aligned with the lip at the distal reamer end.

### BRIEF DESCRIPTION OF THE DRAWINGS

Further advantages and features of the invention can be gathered from the claims and the following description of two preferred embodiments of the inventive facet joint reamer and with reference to the attached drawings, wherein show:

FIG. 1 A first development of the inventive facet joint reamer, partly in longitudinal section.

FIG. 2 Another development of an inventive facet joint reamer, also partly in longitudinal section.

FIG. 3 A front view of the insertion opening of the handle of an inventive facet joint reamer.

FIG. 4 A rear view of two adjacent lumbar vertebrae of the human vertebral column.

FIG. 5 A part sectional view of the spinal intervertebral disks between two vertebrae according to FIG. 4.

### DETAILED DESCRIPTION OF THE DRAWINGS

The facet joint reamer 1 according to the invention has a substantially cylindrical steel shank 2, which is provided with teeth 4 at its distal end 3. At the proximal end 5 the shank has a fastening part 6 in the form of a polygon, here trihedron plastic sleeve injection moulded onto the same and whose side wall is provided with a depression 7. Shank 2 is nondestructively releasably connectable to a handle 11.

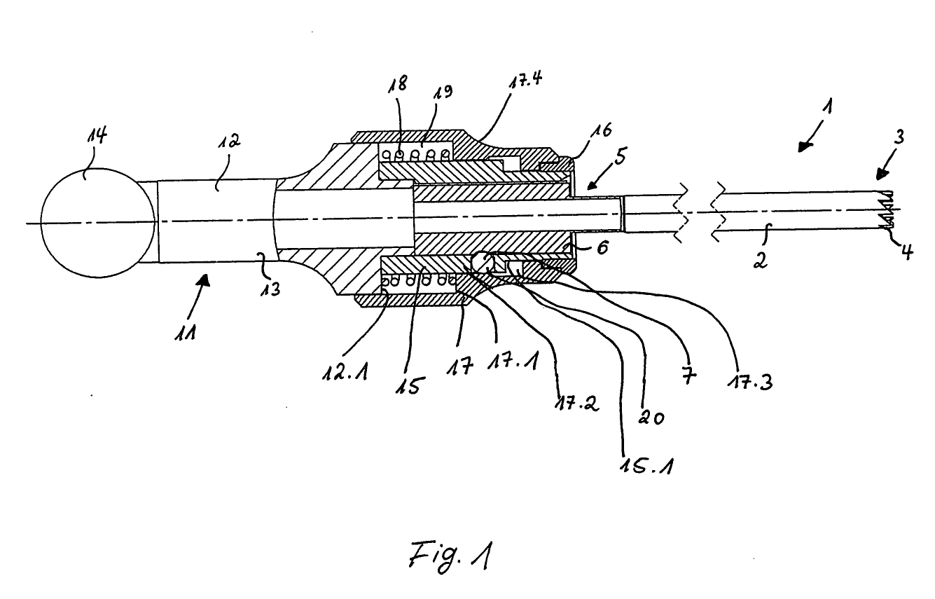

Handle 11 has a T-shaped handle part 12 with a longitudinal bar 13 and at its distal end a crossbar 14. To the handle part 12, more precisely the proximal end of longitudinal bar 13, is connected in fixed manner a sleeve 15, on whose end rests a seal 16. Along the sleeve can be axially slid counter to the action of a spring 18 a slider 17, said spring being placed in a recess 19 between sleeve 15 and slider 17 and its abutment in the represented embodiment of FIG. 1 is on the one hand, proximally, on a frontal ring section 12.1 of handle part 12 and on the other on a ring-shoulder 17.1 of slider 17, so that the spring preloads or presses it in the distal direction.

A recess of sleeve 15 contains a ball 20, which is held on the radial inside by an annular flange 15.1 bounding the recess, so that it cannot drop out of the sleeve 15. Slider 17 has an area 17.2 sliding on the sleeve 15 and by means of which, as shown in FIG. 1, the sleeve can be pressed into recess 7 of part 6. Following onto the same on the side remote from the sleeve is provided a recess 17.3, which has larger radial dimensions than wall 17.2 and consequently, if area 17.3 reaches the axial height of the ball, releases the latter, so that the shank 2 can be extracted from the handle 11.

On the side of slider 17 remote from the cross-section 14 of handle 11 is provided a gripping trough 17.4. Counter to the action of spring 18 and by action on gripping trough 17.4 and pulling the slider 4, the latter can be moved counter to cross-section 14 and then the widened recess 17.3 of the slider arrives at the axial height of ball 20 and in this way releases the same for removing or inserting reamer 2. On releasing the slider 17 again, under the influence of spring 18 its area 17.2 slides over ball 20, presses the latter into recess 7 and in this way locks reamer 2 in the axial position represented here.

As a result of the polygonal construction of connecting. part 6 and the recess of handle 11, the reamer is also connected to rotate in handle 11.

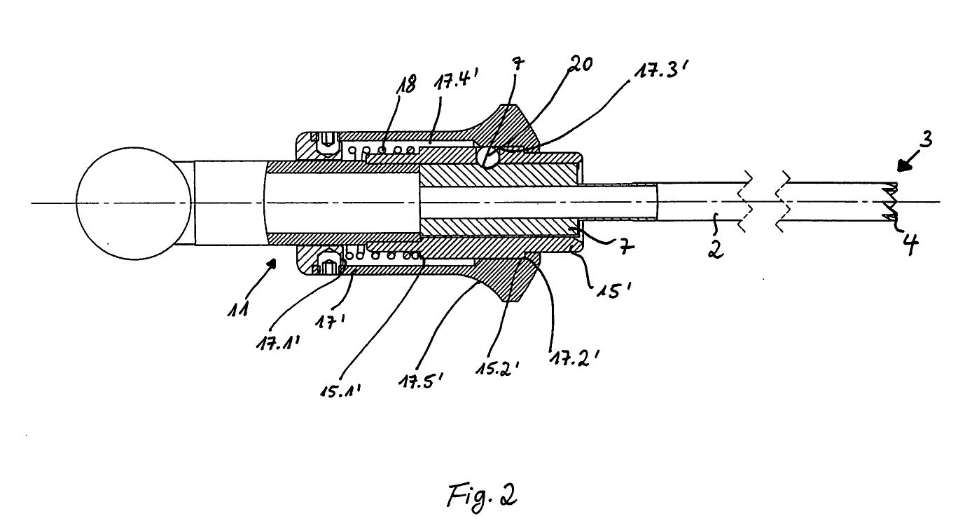

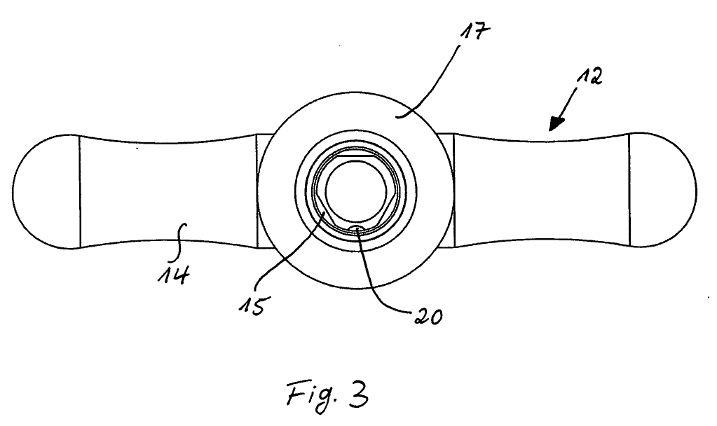

FIG. 2 shows another development of the inventive reamer 1, in which identical parts carry the same reference numerals. For a description thereof reference should be made to the preceding statements and only the differences are explained in detail hereinafter.

Also in the case of the development of FIG. 2 a sleeve 15′ as provided on handle 11 and is surrounded by a slider 17′ slidable axially along the same. At the rear, proximal end of sleeve 15′ is formed a shoulder 15.1′ and once again a helical spring 18 is provided with an inwardly projecting annular step 17.1 formed proximally on slider 17′. Said spring moves the slider 17rearwards or in the proximal direction. The slider has an abutment by means of cooperating shoulders 15.2′ on sleeve 15′ and 17.2′ on the slider..

A ball 20 is seated in the described manner in a recess of sleeve 15′. Slider 17′ has an area 17.3′, whose internal diameter is aligned in this area with the external diameter of sleeve 15. A widened slider area 17.4′ is provided proximally to area 17.3′.

Spring 18 presses the slider into the position shown in FIG. 2, in which area 17.3′ of slider 17′ is at the axial height of ball 20 and presses the latter into recess 7 of connecting part 6 and thus locks shank 2 in handle 11.

On the slider is also formed a gripping part 17.5′ with which said slider can be moved in the distal direction.

If the surgeon grasps the gripping area 17.5 and slides slider 17′ in the distal direction, the widened area 17.4′ of slider 17′ arrives at the axial height of ball 20, so that the latter can move radially outwards, passes out of recess 7 and consequently shank 2 can be removed from handle 7 and also in this slider position a shank 2 can be inserted in handle 11.

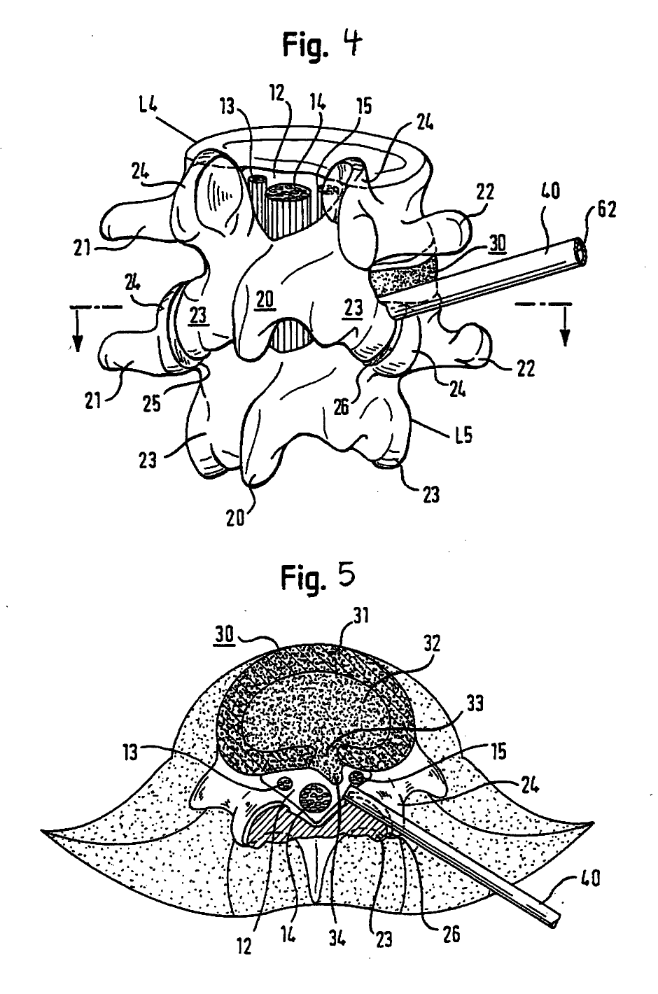

The inventive reamer is used in the manner described hereinafter relative to FIGS. 4 and 5. In exemplified manner FIG. 4 shows the fourth and fifth lumbar vertebrae L4, L5 and between the same an intervertebral disk 30 with a strangulation 33 at the fibrous ring 31 immediately to the right of the central axis of the vertebral column with an extrusion 34 of intervertebral disk pulp tissue into the vertebral canal—FIG. 5.

In the interior of the vertebral canal 22 are diagrammatically shown nerve structures 23, 24, 25. Each vertebra L4, L5 has a spinous process 40,and a left and right-hand. transverse process 42, left and right-hand, lower; joint-forming processes 43 and left and right-hand, upper, joint-forming processes 44, the left and right-hand joints between the upper and lower lumbar vertebrae L4, L5, referred to as the facet joint 46, being formed by in each case the lower processes 43 of the upper vertebra L4 and the upper processes 44 of the lower vertebra L5.

The reamer according to the invention is used in the following way:

Firstly a hollow needle or probe with an external diameter of approximately 1.25 mm is advanced into a position adjacent to the strangulation. A guide wire is then moved through the hollow probe lumen until its distal end projects somewhat over the probe end. The hollow probe is then removed, whereas the guide wire remains in place. A trocar shell is then advanced over the guide wire until the blunt trocar end is located on the facet joint 46. Whilst holding the trocar in this position, a reamer according to the invention with a small diameter of approximately 4.5 mm is advanced over the trocar 54 until the distal reamer end engages on the surface of the facet joint 46. The surgeon now e.g. manually rotates the handle provided at the reamer end close to him, so that a channel is produced in the protuberance 43 of vertebra L4. This step is repeated with larger diameter trocar shells and reamers until an adequate channel diameter is reached to permit the reception of a cannula with a lumen sufficiently large to permit the passage of not only forceps, but also an endoscope. The strangulation is then removed with the forceps, optionally under endoscopic observation.

### REFERENCE NUMERALS LIST

1 Facet joint reamer

2 Shank

3 Distal end

4 Teeth

5 Proximal end

6 Fastening part

7 Depression

11 Handle

12 T-shaped handle part

12.1 Annular section

13 Longitudinal bar

14 Crossbar

15 Sleeve

15.1 Annular flange

15′ Sleeve

15.1′/15.2′ Shoulder

16 Seal

17 Slider

17.1 Annular shoulder

17.2 Wall

17.3 Recess

17.4 Gripping trough

17.5 Gripping area

17′ Slider

17.1′ Annular step

17.2′ Shoulder

17.3′ Area

17.4′ Widened area

17.5′ Gripping part

18 Helical spring

19 Recess

20 Ball

22 Vertebral canal

23, 24, 25 Nerve structures

30 Intervertebral disk

31 Fibrous ring

33 Strangulation

34 Extrusion

40 Spinous process

42 Transverse process

43/44 Processes

46 Facet joint

54 Trocar

L4, L5 Lumbar vertebrae

1. Facet joint reamer with a substantially cylindrical shank, with teeth at the distal shank end and with a handle at the proximal end, wherein shank and handle are separable.

2. Reamer according to claim 1, wherein shank and handle are nondestructively separable.

3. Reamer according to claim 1, wherein shank and handle are provided with a cooperating connecting device.

4. Reamer according to claim 1, wherein at its proximal end the shank has a lateral depression in which engages a ball seated on handle.

5. Reamer according to claim 1, wherein a slider is movable between a release position for releasing the shank from the handle and a locking position in which the shank is reliable held in handle.

6. Reamer according to claim 5, wherein the slider is spring loaded.

7. Reamer according to claim 6, wherein the slider is held in the locking position by a spring.

8. Reamer according to claim 1, wherein the slider has a widened inner area.

9. Reamer according to claim 1, wherein the slider can be slid from its locking position into its release position by manual action counter to the force of spring.

10. Reamer according to claim 1, wherein the slider can be slid in the proximal direction into its release position counter to the action of spring.

11. Reamer according to claim 1, wherein the slider can be slid in the distal direction into its release position counter to the action of spring.

12. Reamer according to claim 1, wherein the shank and handle are connectable to rotate with one another.

13. Reamer according to claim 12, wherein a connecting part of shank is constructed as an external polygon, particularly trihedron and a reception area of handle as an internal polygon, preferably internal trihedron.

14. Reamer according to claim 1, wherein a connecting part of the shank is made from plastic.
